(19) United States
(12) Patent Application Publication
(10) Pub. No.: US 2008/0254445 A1
(43) Pub. Date: Oct. 16, 2008

(54) Prognosis in Cancer Patients Vaccinated with a Cancer Antigen Peptide-Associated Agent

(76) Inventor: Kyogo Itoh, Saga-ken (JP)

Correspondence Address: WENDEROTH, LIND & PONACK, L.L.P., 2033 K STREET N. W., SUITE 800, WASHINGTON, DC, 20006-1021, US

(21) Appl. No.: 10/571,908

(22) PCT Filed: Sep. 22, 2003
(86) PCT No.: PCT/JP03/12057

§ 371 Date: Mar. 14, 2006

(51) Int. Cl. C12Q 1/68; C12Q 1/02

(52) U.S. Cl. 435/6; 435/29

(57) ABSTRACT

The correlation of clinical benefits and immune responses to peptides in cancer patients who were vaccinated with CTL-directed peptides is shown. The invention relates to a process for determining prognosis in a cancer patient vaccinated with a cancer antigen peptide-associated agent, which comprises measuring a level of an antibody specific to the cancer antigen peptide, and assessing whether the level is significantly increased as compared to the level at pre-vaccination.

### TECHNICAL FIELD

The present invention relates to humoral responses to peptides that correlate with overall survival in advanced cancer patients vaccinated with peptides based on pre-existing peptide-specific cellular responses.

### BACKGROUND ART

Recent advances in tumor immunology has allowed the identification of a number of antigens and epitopic peptides capable of inducing tumor-reactive cytotoxic T lymphocytes (CTLs).¹⁻¹⁴ Some of these peptides were used for clinical trials, but these initial trials obtained rare clinical responses as well as dim levels of immune responses to peptides.¹⁵⁻²⁰ One reason for this failure could be an insufficient induction of anti-tumor responses in these regimens, in which peptide-specific memory T cells were not measured in pre-vaccination peripheral blood mononuclear cells (PBMCs). The other reason for failure might in part be due to a lack of an appropriate laboratory marker either to measure immune responses or to predict clinical responses. Regardless of the extensive studies, there are few reproducible and appropriate laboratory markers for prediction of clinical benefits in recently developing peptide-based therapies,¹⁵⁻²³ or in the other types of immunotherapies.²⁴⁻²⁶

Delayed type hypersensitivity (DTH) response is a simple method with high reproducibility, and has often been used a laboratory marker to monitor immune responses in vivo for vaccination against infectious diseases and also malignant diseases. However, controversial results have been obtained regarding DTH response as a laboratory marker for either measuring immune responses to antigens or in the prediction of clinical benefits for vaccinated patients.^{15-27,29,30} As shown in Example hereinafter, DTH response did not correlate with either clinical course or overall survival. In addition, measurements of increased cellular immunity to either peptide or tumor cells did not correlate with overall survival. Collectively, none of the assays for cellular immunity correlated closely with overall survival, regardless of the fact that the vaccinated peptides were screened by CTL precursor assay in pre-vaccination PBMCs. There may be several reasons for this unexpected result. One of them could have to do with reproducibility. CTL precursor frequency analysis, elispot assay, and cytotoxicity assays are generally used as laboratory markers to measure cellular immune responses to vaccinated peptides.^{15-30,33} Although these assays are well-established monitoring systems, none of them is highly reproducible mainly because the CTL precursors in PBMCs are usually very low and the precursor frequency is between 1/1,000 to 1/100,000, whereas the limit of sensitivity of these assays for detection is around 1/3,000 to 1/10,000 cells.^{15-30,32-34} The lower reproducibility of these cellular assays might also be due to in vitro biases, including the cells' condition of cryopreservation, the culture medium, culture conditions, and the numbers and viability of cells at the time of harvesting. In contrast, the measurement of humoral immune responses can be relatively reproducible since antibody molecules reactive to peptides are generally stable and abundant in serum samples.^{21-23,31-32} One of the other reasons is that these CTL assays use PBMCs, not tumor-infiltrating lymphocytes, and thus do not necessarily reflect the CTL activity at tumor sites. It is well known that T cells in the circulation rarely infiltrate into tumor sites. In contrast, IgG molecules might easily reach either peri-tumor sites or intra-tumor sites. This assumption is in part supported by the recent observation that inflammatory responses were observed around prostate cancers at the time of surgery in patients who received peptide vaccinations based on information regarding antibodies reactive to peptides before radical prostatectomy (Noguchi et al. unpublished result).

### DISCLOSURE OF THE INVENTION

In accordance with the present invention, the inventor investigated the correlation of clinical benefits and immune responses to peptides in HLA-A24-positive or -A2-positive cancer patients who were vaccinated with these CTL-directed peptides, and reported that humoral responses to peptides correlated with overall survival.

The invention is directed to the finding of a laboratory marker for overall survival in advanced cancer patients who were vaccinated with peptides based on pre-existing peptide-specific cytotoxic T lymphocyte (CTL) precursors in the circulation.

Thus, the present invention relates to:
(1) A process for determining prognosis in a cancer patient vaccinated with a cancer antigen peptide-associated agent, which comprises collecting an sample of blood serum from the patient, measuring a level of an antibody specific to the cancer antigen peptide, and assessing whether the level is increased, wherein the prognosis is determined as a good one when the level of the antibody is increased.
(2) The process of (1), wherein the cancer antigen peptide-associated agent is selected from a group consisting of a cancer antigen protein, a cancer antigen peptide thereof, a gene thereof, and a derivative of their substances.
(3) The process of (1) or (2), wherein a cancer patient is an advanced cancer patient.
(4). A process for collecting a data for determining prognosis in a cancer patient vaccinated with a cancer antigen peptide-associated agent, which comprises collecting an sample of blood serum from the patient, measuring a level of an antibody specific to the cancer antigen peptide, and assessing whether the level is increased so as to arrange the data, wherein the prognosis is determined as a good one when the level of the antibody is increased.
(5) The process of (4), wherein the cancer antigen peptide-associated agent is selected from a group consisting of a cancer antigen protein, a cancer antigen peptide thereof, a gene thereof, and a derivative of their substances.
(6) The process of (4) or (5), wherein a cancer patient is an advanced cancer patient.
(7) A data as prepared according to the process of any one of (4) to (6).
(8) Use of an antibody specific to a cancer antigen peptide for determining prognosis in a cancer patient vaccinated with a cancer antigen peptide-associated agent.

### BRIEF DESCRIPTION OF DRAWINGS

FIG. 1 depicts the graphs showing overall survival and laboratory markers.

### BEST MODE FOR CARRYING OUT THE INVENTION

In an embodiment, the invention provides a process for determining prognosis in a cancer patient vaccinated with a cancer antigen peptide-associated agent.

The term “prognosis” as used herein means a forecast as to the probable outcome of a disease, and the prospect as to recovery from a disease as indicated by the nature and symptoms of the case. Typically, the prognosis is evaluated as overall survival, median survival time (MST), and/or one-year survival rate.

“Overall survival” means a survival time of patients after vaccination. “Median survival time (MST)” means a median value of the overall survival of patients in each group. For example, in case of a group consisting of 9 patients, MST is an overall survival of the fifth patient (the median patient), when the 9 patients are arranged according to the survival time. MST is often used since an average of survival time may be deviated from a suitable value of the survival time when the average is calculated from the group comprising a patient having a extremely long or short survival time. “One-year survival rate” means a survival time one year after the vaccination. When 3 of 10 patients who were vaccinated are survival one year after the vaccination, then one-year survival rate is 30%. No correlation between one-year survival rate and overall survival or MST was occasionally observed.

The term “cancer antigen peptide-associated agent” as used herein means a tumor antigen protein and the gene thereof, a tumor antigen peptides derived from the tumor antigen protein and the gene thereof, and a derivative of their substances. Tumor antigen peptides are generated by degradation of tumor antigen proteins, which are proteins specific for tumors, in cells with proteasomes, which peptides are intracellularly synthesized. The tumor antigen peptides thus generated bind to MHC class I antigens (HLA antigens) in endoplasmic reticulum to form complexes, and the complexes are transported to the cell surface to be presented as an antigen.

Tumor antigen proteins as used herein include a protein named MAGE from human melanoma cells (Science, 254:1643, 1991); melanosomal proteins such as a melanocytic tissue-specific protein, gp100 (J. Exp. Med., 179:1005, 1994), MART-1 (Proc. Natl. Acad Sci. USA, 91:3515, 1994), and tyrosinase (J. Exp. Med., 178:489, 1993); MEGE-related proteins (J. Exp. Med., 179:921, 1994); β-catenin having a tumor-specific amino acid mutation (J. Exp. Med., 183:1185, 1996); and CDK4 (Science, 269:1281, 1995); HER2-neu (J. Exp. Med., 181:2109, 1995), p53 (variant) (Proc. Natl. Acad. Sci. USA, 93:14704, 1996); tumor markers such as CEA (J. Natl. Cancer Inst., 87:982, 1995), PSA (J. Natl. Cancer Inst., 89:293, 1997); and viral proteins such as HPV (J. Immunol., 154:5934, 1995) and EBV (Int. Immunol., 7:653, 1995). Detailed descriptions of these substances can be found in published reviews (e.g. Immunol. Today, 18:267, 1997; J. Exp. Med., 183:725, 1996; and Curr. Opin. Immunol., 8:628, 1996).

Typical examples of tumor antigen peptides as used herein include, but not limited to, tumor antigen peptides described in WO97/46676, WO99/29715 and WO99/33977; tumor antigen peptides derived from cyclophilin B (WO99/67288); tumor antigen peptides derived from SART-1 (WO00/06595); tumor antigen peptides derived from SART-3 (WO00/12701); tumor antigen peptides derived from ART-1 (WO00/32770); tumor antigen peptides derived from SART2 (J. Immunol., 164:2565, 2000); tumor antigen peptides derived from lck (Eur. J. Immunol., 31:323, 2001); tumor antigen peptides derived from ART4 (Cancer Res., 60:3550, 2000); and tumor antigen peptides derived from ppmAPkk, WHSC2, UBE2V, HNRPL, EIF (Cancer Res., 61:2038, 2001).

Genes as used herein of a cancer antigen protein and a cancer antigen peptide can be prepared according to the well-known method such as those described in for example Molecular Cloning 2nd Edt. Cold Spring Harbor Laboratory Press (1989) with consulting the references as described above.

Derivatives as used herein of a cancer antigen protein, a cancer antigen peptide and a gene thereof mean artificial proteins and peptides that are prepared on the basis of the amino acid sequence of the cancer antigen protein and peptide, as well as genes thereof. Typical examples of derivatives include a protein and a peptide which have an amino acid sequence having a substitution, a deletion and/or an addition of a few amino acid residue in the amino acid sequence of naturally-occurring cancer antigen proteins and peptides, and which have a similar activity for inducing immunoresponses as the naturally-occurring cancer antigen proteins and peptides.

In accordance with the invention, to determine prognosis in a cancer patient vaccinated with a cancer antigen peptide-associated agent, an sample of blood serum is first collected from the patient. Then, a level of an antibody specific to the cancer antigen peptide is measured. Even when any substance as described above is vaccinated as a cancer antigen peptide-associated agent, an antibody specific to a cancer antigen peptide can be measured to determine prognosis. Measurement of the level of antibody could be performed by means of any one of the well-known methods such as ELISA.

In the assessment of the level of the antibody, when the level is significantly increased compared to that at pre-vaccination, then the prognosis is determined to be good. Usually, the level of the antibody is determined in triplicate, and the mean and the standard deviation are calculated. These values are statistically compared between before and after the vaccination. When p value is less than 0.05 (p<0.05), then it will be considered significant.

A data for determining prognosis in a cancer patient vaccinated with a cancer antigen peptide-associated agent, which is prepared according to the process of the invention can be translated into digitized information such as a database. Such database could be readily utilized when put into a floppy disk or a hard disk of a computer. In this connection, the present invention encompasses the data as prepared according to the process of the invention, database derived from the data, and a floppy disk and a hard disk of a computer containing the data.

### EXAMPLES

The present invention is further illustrated by the following examples, but is not limited by these examples in any respect.

### Example 1

### Patients and Method

### Trial Eligibility

The ethical review boards of the Kurume University School of Medicine, and the Hokkaido University School of Medicine approved the study protocol. Complete written informed consent was obtained from all patients at the time of enrollment. According to the protocol, patients were required to be HLA-A24-positive and/or HLA-A2-positive, to have a histologically confirmed lesion of a malignant tumor, to have been untreated for at least 4 weeks prior to the study, and to have an Eastern Cooperative Oncology Group (ECOG) performance status of 0 to 2. Eligibility criteria included an age from 20 to 85 years, a creatinine level less than 1.4 mg/dl, a bilirubin level less than 1.5 mg/dl, platelet count of more than 100,000/mm³, hemoglobin of more than 8.0 g/dl, and total WBC count of more 3000/mm³. Hepatitis B and C antigens were required to be negative. No patient had received any concurrent treatments, steroids, or any other immunosuppressive drugs for 4 weeks prior to the initial vaccination. This clinical study was carried out from November 2000 through November 2002.

### Patients' Characteristics

One-hundred-thirteen patients with advanced cancer (28 colorectal, 22 prostate, 15 lung, 14 gastric, and 34 other cancers) were enrolled in a phase I clinical study of peptide vaccination in which peptide-specific CTL precursors of pre-vaccination peripheral blood mononuclear cells (PBMCs) were measured followed by vaccination with these peptides (maximum 4). The types of cancer included the following: colorectal cancer (HLA-A24: n=22, HLA-A2: 6), prostate cancer (n=12, 10), lung cancer (n=10, 5), gastric cancer (n=12, 2), melanoma (n=5, 2), cervical cancer (n=4, 2), ovarian cancer (n=1, 2), breast cancer (n=1, 2), esophageal cancer (n=1, 2), uterine cancer (n=2, 0), pancreatic (n=2, 0), liomyosarcoma (n=2, 0), thyroid cancer (n=1, 0), chronic lymphocytic leukemia (n=1, 0), bladder cancer (n=1, 0), renal cell carcinoma (n=1, 0), periurethral cancer (n=0, 1), and seminoma (n=0, 1) (Table 1). The average patient age was 61.0 years (range: 23-85). Patients' performance status evaluated by ECOG criteria was 0 (n=68), 1 (n=32), and 2 (n=13). All the patients showed failure to respond to chemotherapy, hormonal therapy, and/or radiotherapy with clinical stage IV or recurrence.

### Methods for Determining Clinical Laboratory Test Values

For cellular responses, pre- and post (6th)-vaccination PBMCs were provided for measurement of both peptide-specific CTL precursors by IFN-γ-release assay and tumor-reactivity by ⁵¹Cr-release assay. Delayed type hypersensitivity (DTH) was also measured. For humoral response, pre- and post (6th)-vaccination sera were provided for measurement of peptide-reactive IgG by an enzyme-linked immunosorbent assay (ELISA).

### Peptides and Selection for Vaccination

The peptides utilized in the present study were prepared under conditions of Good Manufacturing Practice using the Multiple Peptide System, San Diego, Calif. The peptide sequences are shown in Table 2 hereinafter. These peptides have the ability to induce HLA-A24 or -A2 restricted and tumor-specific CTL activity in the PBMCs of cancer patients.⁵⁻¹⁴ These peptides were dissolved and stored at −80° C. Stock solutions were diluted with saline just before use.

For the peptide screening, prevaccination PBMCs were provided for assays of peptide-specific CTL precursors using methods reported previously.²⁸ Peptide-specific IFN-γ production was calculated by subtraction of IFN-γ production of the peptide-stimulated PBMCs in response to a negative control (HIV peptide) from that in response to a corresponding peptide. According to the results, up to the four positive peptides were selected for each patient, and were vaccinated as the CTL precursor-oriented peptide vaccine, if an immediate-type hypersensitivity reaction against each peptide was not seen in a skin test performed before vaccination. The screening of peptide-specific CTL precursors was also performed by the same method after the 6th vaccination to evaluate the in vivo cellular responses to the peptides. Cellular responses to tumor cells in a HLA-A24 or -A2 restricted manner in pre- and post (6th)-vaccination PBMCs were measured using a standard ⁵¹Cr-release assay whose methods were described elsewhere.^{19,20}

### Clinical Protocol

Skin tests were performed by intradermal injection of 10 μg of each peptide using a tuberculin syringe and a 26-gauge needle. Saline was used as a negative control for assessment of hypersensitivity. Immediate- and delayed-type hypersensitivity (DTH) reactions were determined at 20 min and 24 h after the skin test, respectively. At least 5 mm of induration or 10 mm of erythema read 24 hours after injection was needed in order to score the skin test as positive. If immediate-type hypersensitivity was negative, the peptide was vaccinated into the patients' subcutaneous tissue of the upper arm in cases with lung cancer, lateral abdominal wall in cases with gastric cancer, or anterior thigh with the other cancers. Two ml of the peptide, which was supplied in vials containing 2 mg/ml sterile solution, was mixed with an equal volume of incomplete Freund's adjuvant (IFA) (Montanide ISA-51; Seppic, Paris, France) and emulsified in 5-ml sterilized syringes. Three ml of each prepared peptide emulsion (maximum 4 peptides at one vaccination) were injected three times every two weeks. For patients showing a favorable clinical course, the vaccinations were continued every two to four weeks with informed consent from each patient.

### Detection of Serum IgG Levels

An ELISA was used to detect the serum IgG levels specific to the administered peptides, as reported previously.²⁰⁻²³ Briefly, 100 μl well of serum samples diluted with 0.05% Tween 20-Block Ace was added to the peptide (20 μg/well)-immobilized plate, after which the plate was blocked with Block Ace (Yukijirushi, Tokyo, Japan), and washed. After a 2-hr incubation, the plate was washed and further incubated for 2 hr with a 1:1000-diluted rabbit anti-human IgG (DAKO, Glostrup, Denmark). The plate was washed, and 100 μl of 1:100-diluted goat anti-rabbit Ig-conjugated horseradish peroxidase-dextran polymer (EnVision, DAKO) was then added to each well, and the plate was incubated for 40 min. After washing, 100 μl/well of tetramethyl-benzidine substrate solution (KPL, Guildford, UK) was added, and the reaction was stopped by the addition of 1 M phosphoric acid. In order to estimate peptide-specific IgG levels, the optical density values of each sample were compared with those of serially diluted standard samples, and the values were shown as optical density.

### Evaluation of Clinical Responses and Statistical Analysis

All known sites of disease were evaluated by CT-scan or X-ray examination. Patients were assigned a response category according to the RESPONSE EVALUATION CRITERIA IN SOLID TUMORS (RECIST criteria), the revised version of the WHO criteria published in the WHO Handbook for reporting results of cancer treatment (Geneva, 1979) June 1999 (Final). For prostate cancer patients without measurable lesions, serum prostate-specific antigen (PSA) levels were used as a marker for evaluation, as reported previously.²³

Overall survival was evaluated from the entry date of these clinical trials regardless of peptide vaccinations after phase 1 trials, and was analyzed in order to investigate correlation between clinical benefits and immune responses. Kaplan-Meier curves were described and survivals were compared using the logrank test

### Results

### Vaccinated Peptides and Immune Responses

CTL precursors reacting to peptides were detected in pre-vaccination PBMCs for vaccination; the frequency of vaccinated peptides is given in Table 2. The most frequently used peptide was the SART3₁₀₉ (38 of 78 cases), followed by the lck₂₀₈ (31 cases) in HLA-A24⁺ patients. In HLA-A2⁺ patients, the most frequently used peptide was the MAP₂₉₄ (15 of 35 cases), followed by the MAP₄₃₂ (14 cases). CTL activity was evaluated in post (6th) -vaccination PBMC in order to evaluate cellular immune responses to the vaccinated peptides. Increased cellular responses were most frequently observed when the SART3₃₁₅ peptide was vaccinated (9 of 22 cases, 41%) followed by the SART3₁₀₉ peptide (12 of 31 cases, 39%). Detailed results for each case are summarized in Table 2. CTL activity to HLA-class I-restricted tumor cells was measured by the standard ⁵¹Cr-release assay in pre- and post (3rd and 6th)-vaccination PBMC. Sixteen of 76 cases tested (21%) showed increased HLA-class I-restricted cytotoxicity. DTH response at the site of a skin test during the first to 6th vaccination was most frequently observed when the SART3₃₁₅ peptide was vaccinated (9 of 26 cases of HLA-A24⁺ patients). DTH response was most frequently observed in HLA-A2⁺ patients vaccinated with the lck₄₂₂ peptide (6 of 12 cases), a summary of which is presented in Table 2. Humoral immune responses to the vaccinated peptides were simultaneously measured in both pre- and post (3rd and 6th)-vaccination sera. Increased levels of IgG antibodies reactive to peptides were most frequently observed when the SART3₁₀₉ was vaccinated (19 of 37 cases), as summarized in Table 2. It is of note that the UBE₄₃ peptide induced humoral immune responses in all 5 cases tested.

### Clinical Responses and Prognostic Marker Analysis

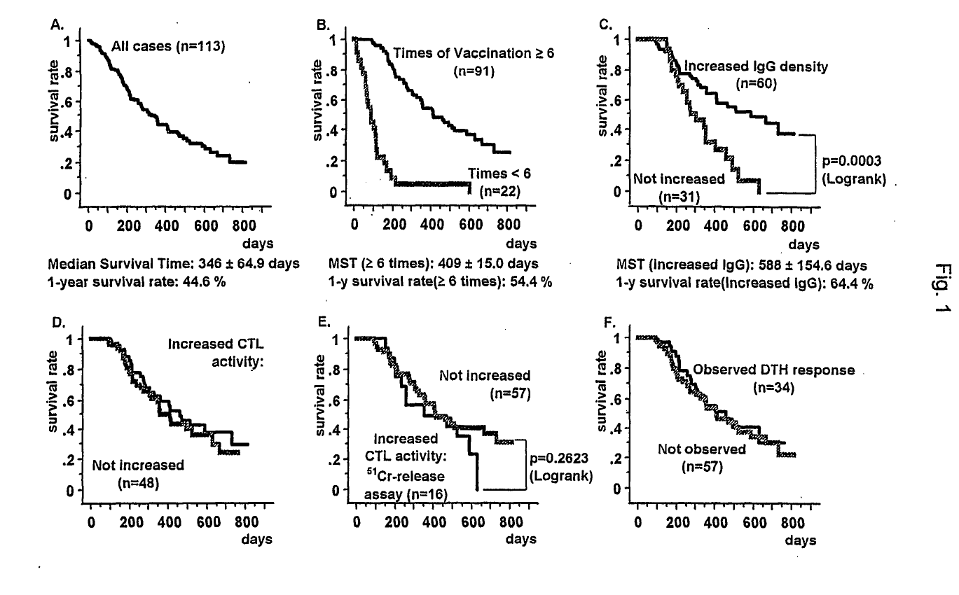

Of 113 cases, 5 cases showed partial response, 2 cases showed minor response, and the remaining 106 cases showed progressive disease. A median survival time (MST) of 113 cases was 346±64.9 (±standard error) days, and a one-year survival rate was 44.6% (FIG. 1A). Twenty-two cases could not achieve one cycle of vaccination (6 times) because of the rapid progression of tumors, while the remaining 91 cases received more than 6 vaccinations. The MST and one-year survival rate of these 91 cases were 409±15.0 days and 54.4%, respectively (FIG. 1B). In patients undergoing more than 6 vaccinations (n=91), 60 cases had detectably increased levels of peptide-specific IgG antibody in their post-vaccination sera against at least one peptide of at maximum 4 vaccination peptides, whereas the remaining 31 cases did not (FIG. 1C). Forty-two among 90 cases tested showed increased CTL activity response to at least one peptide of at maximum 4 vaccination peptides (FIG. 1D), and 16 of 73 cases tested showed increased CTL activity of HLA-class I-restricted cytotoxicity against tumor cells (FIG. 1E) in their post-vaccination PBMCs. Thirty-four of 91 cases showed DTH response to at least one peptide among at maximum 4 vaccination peptides until the 6th vaccination (FIG. 1F). None of cellular responses (peptide-specific CTL precursors, tumor-reactive CTL activity, or DTH reaction) correlated with overall survival. In contrast, the overall survival of patients whose sera showed increased levels of peptide-reactive IgG antibodies (n=60) was more significantly prolonged (p=0.0003) than that of patients whose sera did not show such increased levels (n=31).

### INDUSTRIAL APPLICABILITY

Peptide-specific IgG in post-vaccination sera could be a suitable laboratory maker for the prediction of prolonged survival in advanced cancer patients vaccinated with peptides based on pre-existing CTL precursors.

The feasibility of ELISA as a laboratory marker for monitoring immune responses to vaccinated peptides could be superior to any of the CTL assays from several different points of view. Serum samples. are much easier to preserve than PBMCs. A small amount of sera (10 μl per peptide) is needed for the assay, whereas relatively large numbers of PBMCs (about 10⁶ cells or more per peptide) are needed for CTL assays. The occupation times for ELISA are only one day, whereas CTL assays need 14 to 30 days. Running costs are another advantage of ELISA.

### REFERENCES

1. van der Bruggen P, Traversari C, Chomez P, et al. A gene encoding an antigen recognized by cytolytic T lymphocytes on a human melanoma. Science 254:1643-1647, 1991
2. Kawakami Y, Eliyahu S, Sakaguchi K, et al. Identification of the immunodominant peptides of the MART-1 human melanoma antigen recognized by the majority of HLA-A2-restricted tumor infiltrating lymphocytes. J Exp Med 180:347-352, 1994
3. Brichard V, Van Pel A, Wolfel T, et al. The tyrosinase gene codes for an antigen recognized by autologous cytolytic T lymphocytes on HLA-A2 melanomas. J Exp Med 178:489-495, 1993
4. Peoples G E, Goedegebuure P S, Smith R, et al. Breast and ovarian cancer-specific cytotoxic T lymphocytes recognize the same HER2/neu-derived peptide. Proc Natl Acad Sci USA 92:432-436, 1995
5. Kikuchi M, Nakao M, Inoue Y, et al. Identification of a SART-1-derived peptide capable of inducing HLA-A24-restricted and tumor-specific cytotoxic T lymphocytes. Int J Cancer 81:459-466, 1999
6. Nakao M, Shichijo S, Imaizumi T, et al. Identification of a gene coding for a new squamous cell carcinoma antigen recognized by the CTL. J Immunol 164:2565-2574, 2000
7. Yang D, Nakao M, Shichijo S, et al. Identification of a gene coding for a protein possessing shared tumor epitopes capable of inducing HLA-A24-restructed Cytotoxic T lymphocytes in cancer patients. Cancer Res 59:4056-4063, 1999
8. Gomi S, Nakao M, Niiya F, et al. A cyclophilin B gene encodes antigenic epitopes recognized by HLA-A24-restricted and tumor-specific cytotoxic T lymphocytes. J Immunol 163:4994-5004, 1999
9. Harashima N, Tanaka K, Sasatomi T, et al. Recognition of the Lck tyrosine kinase as a tumor antigen by cytotoxic T lymphocytes of cancer patients with distant metastases. Eur J Immunol 31:323-332, 2000
10. Nishizaka S, Gomi S, Harada K, et al. A new tumor-rejection antigen recognized by cytotoxic T lymphocytes infiltrating into a lung adenocarcinoma. Cancer Res 60:4830-4837, 2000
11. Kawano K, Gomi S, Tanaka K, et al. Identification of a new endoplasmic reticulum-resident protein recognized by HLA-A24-restricted tumor infiltrating lymphocytes of lung cancer. Cancer Res 60:3550-3558, 2000
12. Imai N, Harashima N, Ito M, et al. Identification of Lck-derived peptides capable of inducing HLA-A2-restricted and tumor-specific CTLs in cancer patients with distant metastases. Int J Cancer 94:237-242, 2001
13. Tamura M, Nishizaka S, Maeda Y, et al. Identification of cyclophilin B-derived peptides capable of inducing histocompatibility leukocyte antigen-A2-restricted and tumor-specific cytotoxic T lymphocytes. Jpn J Cancer Res 92:762-767, 2001
14. Ito M, Shichijo S, Tsuda N, et al. Molecular basis of T cell-mediated recognition of pancreatic cancer cells. Cancer Res 61:2038-2046, 2001
15. Jager E, Gnjatic S, Nagata Y, et al. Induction of primary NY-ESO-1 immunity: CD8+ T lymphocyte and antibody responses in peptide-vaccinated patients with NY-ESO-1+ cancers. Proc Natl Acad Sci USA 97:12198-12203, 2000
16. Marchand M, van Baren N. Weynants P, et al. Tumor regressions observed in patients with metastatic melanoma treated with an antigenic peptide encoded by gene MAGE-3 and presented by HLA-A1. Int J Cancer 80:219-230, 1999
17. Rosenberg S A, Yang J C, Schwartzentruber D J, et al. Immunologic and therapeutic evaluation of a synthetic peptide vaccine for the treatment of patients with metastatic melanoma. Nat Med 4:321-327, 1998
18. Valmori D, Dutoit V, Rubio-Godoy V, et al. Frequent cytolytic T-cell responses to peptide MAGE-A10(254-262) in melanoma. Cancer Res 61:509-512, 2001
19. Gohara R, Imai N, Rikimaru T, et al. Phase 1 clinical study of cyclophilin B peptide vaccine for lung cancer patients. J Immunother 25:439-444, 2002
20. Miyagi Y, Imai N, Sasatomi T, et al. Induction of cellular immune responses to tumor cells and peptides in colorectal cancer patients by vaccination with SART3 peptides. Clin Cancer Res 7:3950-3962, 2001
21. Mine T, Gouhara R, Hida N, et al. Immunological evaluation of CTL precursor-oriented vaccines for advanced lung cancer patients. Cancer Sci., 94: 548-556, 2003
22. Tanaka S, Harada M, Mine T, et al. Peptide vaccination for patients with melanoma and other types of cancer based on pre-existing peptide-specific cytotoxic T lymphocyte precursors in the periphery. J. Immunother., 26: 357-366, 2003
23. Noguchi M, Kobayashi K, Suetsugu N, et al. Induction of cellular and humoral immune responses to tumor cells and peptides in HLA-A24 positive hormone-refractory prostate cancer patients by peptide vaccination. Prostate, 57:80-92, 2003
24. Berd D, Maguire H C Jr, Mastrangelo M J: Induction of cell-mediated immunity to autologous melanoma cells and regression of metastases after treatment with a melanoma cell vaccine preceded by cyclophosphamide. Cancer Res 46:2572-2577 1986
25. Kirkwood J M, Ibrahim J G, Sosman J A, et al. High-dose interferon alfa-2b significantly prolong relapse-free and overall survival compared with the GM2-KLH/QS-21 vaccine in patients with resected stage IIb-III melanoma: Results of intergroup trial E1694/S9512/C509801. J Clin Oncol 19:2370-2380, 2001
26. Salgia R, Lynch T, Skarin A, et al. Vaccination with irradiated autologous tumor cells engineered to secrete granulocyte-macrophage colony-stimulating factor augments antitumor immunity in some patients with metastatic non-small-cell lung carcinoma. J Clin Oncol 21:624-630, 2003
27. Reynolds S R, Zeleniuch-Jacquotte A, Shapiro R L, et al. Vaccine-induced CD8+ T-cell responses to MAGE-3 correlate with clinical outcome in patients with melanoma. Clin Cancer Res 9:657-662, 2003.
28. Maeda Y. Hida N, Niiya F, et al. Detection of peptide-specific CTL-precursors in peripheral blood lymphocytes of cancer patients. Br J Cancer 87:796-804, 2002
29. Gonzalez G, Crombet T, Torres F, et al. Epidermal growth factor-based cancer vaccine for non-small-cell lung cancer therapy. Ann Oncol 14:461-466, 2003
30. DiFronzo L A, Gupta R K, Essner R, et al. Enhanced humoral immune response correlates with improved disease-free and overall survival in American Joint Committee on Cancer stage II melanoma patients receiving adjuvant polyvalent vaccine. J Clin Oncol 20:3242-3248, 2002
31. Ohkouchi S, Yamada A, Imai N, et al. Non-mutated tumor-rejection antigen peptides elicit type-I allergy in the majority of healthy individuals. Tissue Antigens 59:259-272, 2002
32. Kawamoto N., Yamada A, Ohkouchi S, et al. IgG reactive to CTL-directed epitopes of self-antigen is enter lacking or unbalanced in atopic dermatitis patients. Tissue Antigens 61:352-361, 2003
33. Scheibenbogen C, Lee K H, Stevanovic S, et al. Analysis of the T cell response to tumor and viral peptide antigens by an IFN gamma-ELISPOT assay. Int J Cancer 71:932-936, 1997
34. Hida N, Maeda Y, Katagiri K, et al. A simple culture protocol to detect peptide-specific cytotoxic T lymphocyte precursors in the circulation. Cancer Immunol Immunother 51:219-228, 2002

TABLE 1
Patients characteristics and median survival times

| | | | Male/ | | MST ± SE |
|---|---|---|---|---|---|
| Sort of Cancer | n | age (range) | Female | HLA-A24/-A02 | (days) |
| Colorectal cancer | 28 | 57.5 (27-78) | 20/8 | 22/6 | 273 ± 70.9 |
| Prostate cancer | 22 | 68.1 (50-85) | 22/0 | 12/10 | 601 ± 84.1 |
| Lung cancer | 15 | 65.7 (58-74) | 9/6 | 10/5 | 668 ± 201.2 |
| Gastric cancer | 14 | 64.7 (49-78) | 12/2 | 12/2 | 139 ± 48.4 |
| Melanoma | 7 | 60.7 (30-76) | 4/3 | 5/2 | 353 ± 82.5 |
| Cervical cancer | 6 | 50.8 (32-67) | 0/6 | 4/2 | 324 ± 157.3 |
| Ovarian cancer | 3 | 55.0 (49-59) | 0/3 | 1/2 | 522 ± 344.6 |
| Breast cancer | 3 | 46.3 (38-58) | 0/3 | 1/2 | — |
| Esophageal cancer | 3 | 63.7 (59-70) | 2/1 | 1/2 | 85 ± 33.1 |
| Uterine cancer | 2 | 61.5 (56, 67) | 0/2 | 2/0 | 415 ± 293.4 |
| Pancreatic cancer | 2 | 61.5 (60, 63) | 1/1 | 2/0 | 165 ± 95.8 |
| Liomyosarcoma | 2 | 57.5 (54, 61) | 0/2 | 2/0 | 61 ± 43.1 |
| Thyroid cancer | 1 | 62 | 0/1 | 1/0 | — |
| Chronic Lymphocytic | 1 | 77 | 1/0 | 1/0 | — |
| Leukemia | | | | | |
| Bladder cancer | 1 | 53 | 1/0 | 1/0 | 199 |
| Renal cell carcinoma | 1 | 39 | 1/0 | 1/0 | — |
| Periurethral cancer | 1 | 55 | 0/1 | 0/1 | — |
| Seminoma | 1 | 23 | 1/0 | 0/1 | 409 |
| Total | 113 | 61.0 (23-85) | 74/39 | 78/35 | 346 ± 64.9 |

TABLE 2
Vaccinated peptide and immune responses

| | | | No. of vaccinated | Increased immune reactions to peptide* | | |
|---|---|---|---|---|---|---|
| | Peptide Name | Sequence | patients | CTL activity | IgG antibody | DTH response |
| HLA-A24-binding | SART1 690 | EYRGFTQDF | 20 | 1/17 (6%) | 2/19 (11%) | 2/20 (10%) |
| | SART2 93 | DYSARWNEI | 19 | 3/15 (20%) | 4/17 (24%) | 0/19 |
| | SART2 161 | AYDFLYNYL | 21 | 2/16 (13%) | 1/18 (6%) | 2/21 (10%) |
| | SART2 899 | SYTRLFLIL | 13 | 0/7 | 2/11 (18%) | 3/13 (23%) |
| | SART3 109 | VYDYNCHVDL | 38 | 12/31 (39%) | 19/37 (51%) | 4/38 (11%) |
| | SART3 315 | AYIDFEMKI | 26 | 9/22 (41%) | 6/25 (24%) | 9/26 (35%) |
| | CypB84 | KFHRVIKDF | 4 | 0/3 | 2/3 (67%) | 0/4 |
| | CypB91 | DFMIQGGDF | 19 | 0/17 | 1/17 (6%) | 1/19 (5%) |
| | Ick208 | HYTNASDGL | 31 | 7/25 (28%) | 9/29 (31%) | 6/31 (19%) |
| | Ick486 | TFDYLRSVL | 25 | 4/21 (19%) | 7/22 (32%) | 6/25 (24%) |
| | Ick488 | DYLRSVLEDF | 26 | 3/21 (14%) | 1/24 (4%) | 5/26 (19%) |
| | ART1 170 | EYCLKFTKL | 9 | 0/7 | 2/7 (29%) | 0/9 |
| | ART4 13 | AFLRHAAL | 4 | 0/1 | 0/2 | 0/4 |
| | ART4 75 | DYPSLSATDI | 19 | 1/16 (6%) | 0/15 | 1/19 (5%) |
| HLA-A02-binding | SART3 302 | LLQAEAPRL | 6 | 0/4 | 0/5 | 0/6 |
| | SART3 309 | RLAEYQAYI | 9 | 0/6 | 1/7 (14%) | 0/9 |
| | CypB129 | KLKHYGPGWV | 6 | 0/6 | 2/6 (33%) | 1/6 (17%) |
| | CypB172 | VLEGMEVV | 6 | 1/4 (25%) | 3/5 (60%) | 1/6 (17%) |
| | Ick246 | KLVERLGAA | 7 | 2/7 (29%) | 6/7 (86%) | 0/7 |
| | Ick422 | DVWSFGILL | 12 | 3/11 (27%) | 0/10 | 6/12 (50%) |
| | MAP294 | GLLFLHTRT | 15 | 1/12 (8%) | 8/15 (53%) | 7/15 (47%) |
| | MAP432 | DLLSHAFFA | 14 | 3/11 (27%) | 5/13 (38%) | 4/14 (29%) |
| | WHSC103 | ASLDSDPWV | 8 | 0/6 | 1/8 (13%) | 1/8 (13%) |
| | WHSC141 | ILGELREKV | 8 | 1/4 (25%) | 2/5 (40%) | 0/8 |
| | UBE43 | RLQEWCSVI | 6 | 1/5 (20%) | 5/5 (100%) | 2/6 (33%) |
| | UBE85 | LIADFLSGL | 2 | 1/2 (50%) | 1/2 (50%) | 0/2 |
| | UBE208 | ILPRKHHRI | 1 | 0/1 | 1/1 (100%) | 0/1 |
| | HNRPL140 | ALVEFEDVL | 5 | 0/5 | 0/5 | 0/5 |
| | HNRPL501 | NVLHFFNAPL | 13 | 1/10 (10%) | 3/13 (23%) | 2/13 (15%) |
| | EIF51 | RIIYDRKFL | 1 | 0/1 | 1/1 (100%) | 0/1 |

*No. of tested cases and % positive in parentheses are shown.

1. A process for determining prognosis in a cancer patient vaccinated with a cancer antigen peptide-associated agent, which comprises collecting an sample of blood serum from the patient, measuring a level of an antibody specific to the cancer antigen peptide, and assessing whether the level is increased, wherein the prognosis is determined as a good one when the level of the antibody is increased.

2. The process of claim 1, wherein the cancer antigen peptide-associated agent is selected from a group consisting of a cancer antigen protein, a cancer antigen peptide thereof, a gene thereof, and a derivative of their substances.

3. The process of claim 1, wherein the cancer patient is an advanced cancer patient.

4. A process for collecting a data for determining prognosis in a cancer patient vaccinated with a cancer antigen peptide-associated agent, which comprises collecting an sample of blood serum from the patient, measuring a level of an antibody specific to the cancer antigen peptide, and assessing whether the level is increased so as to arrange the data, wherein the prognosis is determined as a good one when the level of the antibody is increased.

5. The process of claim 4, wherein the cancer antigen peptide-associated agent is selected from a group consisting of a cancer antigen protein, a cancer antigen peptide thereof, a gene thereof, and a derivative of their substances.

6. The process of claim 4, wherein the cancer patient is an advanced cancer patient.

7. A data as prepared according to the process of claim 4.

8. (canceled)

9. The process of claim 2, wherein the cancer patient is an advanced cancer patient.

10. The process of claim 5, wherein the cancer patient is an advanced cancer patient.
